(19) United States
(12) Patent Application Publication
(10) Pub. No.: US 2003/0220391 A1
(43) Pub. Date: Nov. 27, 2003

(54) Pharmaceutical compositions of orally active taxane derivatives having enhanced bioavailability

(76) Inventors: Joseph Ballard Bogardus, Princeton, NJ (US); Robert Kevin Perrone, Belle Mead, NJ (US); Krishnaswamy Srinivas Raghavan, Cranbury, NJ (US); Sailesh Amilal Varia, Princeton Junction, NJ (US)

Correspondence Address: Stephen B. Davis, Bristol-Myers Squibb Company, Patent Department, P.O. Box 4000, Princenton, NJ, 08543-4000, US

(21) Appl. No.: 10/323,551

(22) Filed: Dec. 19, 2002

Related U.S. Application Data
(60) Provisional application No. 60/342,889, filed on Dec. 20, 2001.

(51) Int. Cl. A61K 31/337

(52) U.S. Cl. 514/449

(57) ABSTRACT

Disclosed are pharmaceutical compositions which comprise an orally-active taxane derivative and a pharmaceutically acceptable solubilizing agent, and which provide effective and consistent oral absorption of the taxane derivative.

This application claims priority benefit under Title 35 §119(e) of United States provisional Application No. 60/342,889 filed Dec. 20, 2001. The present invention relates to pharmaceutical compositions of orally effective taxane derivatives and to their use for inhibiting tumor growth in mammalian hosts. The compositions of the invention enable the production of dosage units that afford sufficient and consistent absorption of the taxane derivative, thereby providing safe and effective antitumor treatment.

### FIELD OF THE INVENTION

### BACKGROUND OF THE INVENTION

Taxanes are diterpene compounds having demonstrated antineoplastic activity. Taxanes such as paclitaxel (Taxol®) and docetaxel (Taxotere®), a semi-synthetic analog of paclitaxel, are clinically useful antitumor agents which impart a cytotoxic effect in vivo by a mechanism involving abnormal polymerization of tubulin and disruption of mitosis.

These agents are commercially available in formulations adapted for intravenous administration. The antitumor activity of taxanes is highly schedule dependent, and can be enhanced by prolonged exposure of tumors to the antitumor agents. Oral dosing of taxanes is a strategy that is being pursued to fully exploit the potential therapeutic advantages afforded by this route of administration. These treatment regimens could include prolonged treatment at or near the maximum tolerated dose to maximize the cytotoxic effect, and chronic metronomic dosing below the maximum tolerated dose to synergistically utilize the anti-angiogenic properties of the drug, while maintaining some cytotoxic effect and possibly reduce the occurrence of drug resistance in the tumors.

Because a number of studies have shown that the oral activity of paclitaxel is essentially nil, methods for administering taxanes in the presence of modulators have been investigated as a means of increasing the amount of taxanes in the plasma after oral administration. The literature provides reports of increases in systemic exposures of paclitaxel and docetaxel following oral administration of these antitumor agents as their intravenous solution formulations co-administered with known (pgp) efflux inhibitors, such as cyclosporin A (S. Broder, et al, U.S. Pat. No. 5,968,972, Oct. 19, 1999; J. V. Asperen et al, “Enhanced Oral Absorption and Decreased Elimination of Paclitaxel in mice Cotreated with Cyclosporin A”, Clinical Cancer Research, October 1998, Vol. 4, 2293-2297; J. M. Terwogt, et al, Lancet, “Co-Administration of cyclosporin enables oral therapy with paclitaxel”, 1998, Vol. 352, pg 285; J. M. Terwogt et al, Clinical Cancer Research, “Co-Administration of Oral Cyclosporin A Enables Oral Therapy with Paclitaxel”, November 1999, Vol. 5, pg 3379-3384; C. D. Britten et al, “Oral Paclitaxel and Concurrent Cyclosporin A: Targeting Clinically Relevant Systemic Exposure to Paclitaxel”, September 2000, Vol. 6, pg 3459-3468; L. J. Denis et al, “Bioavailability of Oral Paclitaxel and Concurrent Cyclosporin A: A Dose Escalation and Feasibility Study”, Proceedings of the American Society of Clinical Oncologists, 35th Annual Meeting, May 15-18, 1999; M. M. Malingre, et al, “Clinical Pharmacology of Oral Paclitaxel in a Dose Escalating Study”, Proceedings of the American Society of Clinical Oncologists, 35^{th} Annual Meeting, May 15-18, 1999; D. J. Richel et al, “Cyclosporin A Strongly Enhances the Oral Bioavailability of Docetaxel in Cancer Patients”, Proceedings of the American Society of Clinical Oncologists, 35^{th} Annual Meeting, May 15-18, 1999). See also published international patent application WO 98/53811 of Baker Norton Pharmaceuticals, Inc. These modulator-containing formulations may also include a solvent, e.g. a polyalkoxylated castor oil, as described in published international patent applications WO 97/15269 and WO 01/30448, both of Baker Norton Pharmaceuticals, Inc. Although reports involving human clinical trials presented plasma levels of taxanes orally dosed in this manner, several disadvantages of this method of dosing were also described, including unpleasant taste, emesis, high interpatient variability, and non-linear response in absorption versus dose.

A desire for increased bioavilability of taxanes upon oral administration, while avoiding the above-noted drawbacks of modulators such as cyclosporins, provided a stimulus for the preparation of orally-effective analogs. One such class of taxane analogs is disclosed in WO 01/56565. The taxane analogs described in WO 01/56565, having the general formula I, shown below, display a significant inhibitory effect with regard to abnormal cell proliferation and have therapeutic properties that make it possible to treat patients who have pathological conditions associated with an abnormal cell proliferation. In addition, these compounds possess significant oral bioavailability, and thus can elicit their positive therapeutic affects after oral administration.

Oral pharmaceutical compositions containing taxanes (e.g. paclitaxel or docetaxel) at least 30 weight % of a taxane carrier, having an hydrophile/lipophile balance (HLB) of at least about 10, and 0-70 weight % of a viscosity reducing co-solubilizer are disclosed in published international application WO 00/78247 of Baker Norton Pharmaceuticals, Inc.

The development and therapeutic usefulness of such orally active taxane analogs as antitumor agents depends to a large extent on the attainment of formulations that provide not only suitable oral bioavailability, but also acceptable inter- and intra-patient variability in the extent of absorption. Parameters affecting the bioavailability of a drug following oral administration include water solubility, drug absorption in the GI tract, and first-pass effect. In the case of drugs having poor aqueous solubility, such as paclitaxel and docetaxel, drug absorption is often dissolution rate-limited and, therefore, dosage forms in which the drug is solubilized typically provide the best oral bioavailability. However, it is generally preferred to have a solid dosage form for improved patient compliance, taste masking and other factors.

Thus, there exists an unmet need for chemically and physically stable dosage forms of orally effective taxanes, and especially solid dosage units, which allow for convenient dosing and which afford effective and consistent oral absorption.

### SUMMARY OF THE INVENTION

According to one aspect of the present invention, there is provided a pharmaceutical composition comprising an antitumor effective amount of an orally-active taxane derivative of Formula I or II:

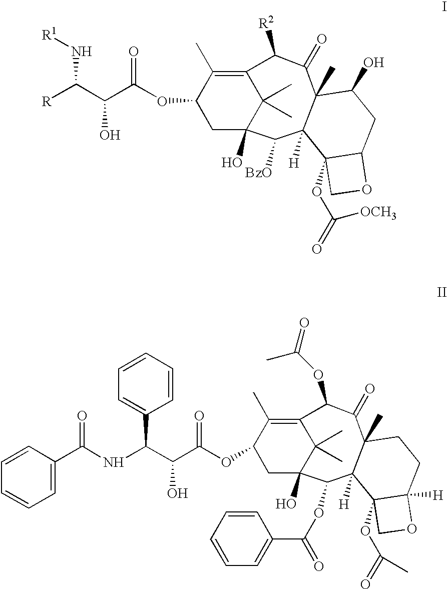

wherein R is phenyl, isopropyl, or tert butyl, R¹ is —C(O)R^{Z} in which R^{Z} is (CH₃)₃CO—, (CH₃)₃CCH₂—, CH₃(CH₂)₃O—, cyclobutyl-, cyclohexyloxy, or (2-furyl), and R² is CH₃ C(O)O—, and a pharmaceutically acceptable solubilizing agent for the taxane derivative of Formula I or II.

The solubilizing agent preferably consists essentially of at least one of the following solubilizer compounds: (a) a polyether glycol, (b) a saturated or unsaturated polyglycolized glyceride, or (c) a solid amphiphilic surfactant and optionally, further includes (d) an alcohol other than a polyether glycol, (e) a fatty acid ester derivative of a polyhydric alcohol, (f) a surfactant other than (c), (g) a vegetable oil, and (h) a mineral oil, or a mixture of any of (d)-(h).

According to another aspect of this invention, there is provided a method of inhibiting tumor growth in a mammalian host which comprises administering to the host, preferably orally, a tumor-growth inhibiting amount of the above-described composition.

As will appear from the examples provided below, the pharmaceutical compositions of the invention, which include both solution and encapsulated semi-solid dosage forms of a taxane derivative of Formula I or II, above, are pharmaceutically acceptable, chemically and physically stable and provide effective and consistent oral absorption.

### DETAILED DESCRIPTION OF THE INVENTION

The preparation of the compounds of Formula I, above, is set forth in detail, along with the manner of using such compounds as antitumor agents, in WO 01/56565. The Formula II compound is also well known to those skilled in the art.

Preferred embodiments of the compounds of Formula I, including their pharmaceutically acceptable salts, are set forth in Table 1.

TABLE 1
Orally Active C-4 Methyl Carbonate Taxanes

| Compound | R | R¹ | R² |
|---|---|---|---|
| Ia | (CH₃)₃C— | (CH₃)₃COC(O)— | CH₃C(O)O— |
| Ib | (CH₃)₂CH— | (CH₃)₃COC(O)— | CH₃C(O)O— |
| Ic | Phenyl- | (CH₃)₃CCH₂C(O)— | CH₃C(O)O— |
| Id | Phenyl- | CyclobutylC(O)— | CH₃C(O)O— |
| Ie | (CH₃)₃C— | CyclohexylOC(O)— | CH₃C(O)O— |
| If | (CH₃)₃C— | (CH₃)₃CCH₂C(O)— | CH₃C(O)O— |
| Ig | Phenyl- | (CH₃)₃COC(O)— | CH₃C(O)O— |
| Ih | Phenyl- | CH₃(CH₂)₃OC(O)— | CH₃C(O)O— |
| Ij | (CH₃)₃C— | CyclobutylC(O)— | CH₃C(O)O— |
| Ik | (CH₃)₃C— | (2-furyl)C(O)— | CH₃C(O)O— |

Among the compounds listed in Table 1, or pharmaceutically acceptable salts thereof, particularly preferred, are Ia, If, Ij and Ik. Compound Ia, 3′-tert-butyl-3′-N-tert-butyloxycarbonyl-4-deacetyl-3′-dephenyl-3′-N-debenzoyl-4-O-methoxycarbonyl-paclitaxel, is the most preferred compound for use in practicing the present invention.

As previously described, several different types of solubilizers for the taxane derivatives of Formulas I and II may be used for the solubulizing agent in the composition of the invention. Suitable polyether glycols include, without limitation, polyethylene glycol (PEG) and polypropylene glycol. Particularly preferred are PEGs within the molecular weight range from 200-8,000 (commercially available from Union Carbide and BASF, among others), which includes those that are liquid at room temperature (e.g. PEG 200-400) and those that are solid at room temperature (e.g. PEG 600-8,000, and the like). Representative examples of useful saturated, polyglycolized glycerides include, without limitation, Gelucire® 44/14, Gelucire® 50/13, Gelucire® 53/10 and the like, which are solid at room temperature; and Labrasol® and the like, which are liquid at room temperate (all available from Gattefosse Corp., Westwood, N.J.). Suitable unsaturated polyglycolized glycerides include Labrafil® M1944CS and the like (also available from Gattefosse Corp.).

Saturated polyglycolized glycerides, such as Gelucires®, are preferred for use in the composition of the invention. They are prepared by the alcoholysis reaction of natural oils with PEG. The saturated polyglycolized glycerides are mixtures of mono-, di- and tri-glycerides of long-chain (C₈ to C₁₈) fatty acids and polyethylene glycol mono-, di-esters obtained either by partial alcoholysis of hydrogenated vegetable oils using polyethylene glycol of relative molecular weight ranging from 200-2000 (predominantly 1500), or by esterification of saturated fatty acids using polyethylene glycol of relative molecular weight ranging from 200-2000 (predominantly 1500) with glycerol. Gelucires® are amphiphilic materials that are surface active and disperse in aqueous media to form micelles, microscopic globules or vesicles in which the incorporated drug is protected from macroprecipitation during contact with an aqueous environment such as the GI tract.

Gelucires® are identified by their melting point/HLB value, with higher HLB's indicating greater water solubility. The preferred saturated polyglycolized glycerides are further characterized as follows:

Gelucire ® 35/10

| | | |
|---|---|---|
| Hydroxyl value | 70-90 | mg KOH/g (nominally, 74 mg KOH/g |
| Saponification | 120-134 | mg KOH/g (nominally, 134 mg |
| value | | KOH/g) |
| Fatty acid | | |
| composition | | |
| Caprylic acid | 1-7% | (nominally, 2.1%) |
| (C8) | | |
| Capric acid | 1-7% | (nominally, 2.2%) |
| (C10) | | |
| Lauric acid | 31-41% | (nominally, 35.4%) |
| (C12) | | |
| Myristic acid | 7-17% | (nominally, 12.9%) |
| (C14) | | |
| Palmitic acid | 12-22% | (nominally, 20.7%) |
| (C16) | | |
| Stearic acid | 23-33% | (nominally, 26.2%) |
| (C18) | | |

Gelucire ® 44/14

| | | |
|---|---|---|
| Hydroxyl value | 30-50 | mg KOH/g |
| Saponification | 76-90 | mg KOH/g |
| value | | |
| Fatty acid | | |
| composition | | |
| Caprylic acid | 4-10% | |
| (C8) | | |
| Capric acid | 3-9% | |
| (C10) | | |
| Lauric acid | 40-50% | |
| (C12) | | |
| Myristic acid | 14-24% | |
| (C14) | | |
| Palmitic acid | 4-14% | |
| (C16) | | |
| Stearic acid | 5-15% | |
| (C18) | | |

Gelucire ® 46/07

| | | |
|---|---|---|
| Hydroxyl value | 65-85 | mg KOH/g (nominally, 70 mg |
| | | KOH/g) |
| Saponification | 126-140 | mg KOH/g (nominally, 139 mg |
| value | | KOH/g) |
| Fatty acid | | |
| composition | | |
| Caprylic acid | <3% | (nominally, <0.1%) |
| (C8) | | |
| Capric acid | <3% | (nominally, <0.1%) |
| (C10) | | |
| Lauric acid | <5% | (nominally, 0.9%) |
| (C12) | | |
| Myristic acid | <5% | (nominally, 1.4%) |
| (C14) | | |
| Palmitic acid | 40-50% | (nominally, 44%) |
| (C16) | | |
| Stearic acid | 48-58% | (nominally, 52.8%) |
| (C18) | | |

Gelucire ® 50/13

| | | |
|---|---|---|
| Hydroxyl value | 36-56 | mg KOH/g (nominally, 52 mg |
| | | KOH/g) |
| Saponification | 67-81 | mg KOH/g (nominally, 74 mg |
| value | | KOH/g) |
| Fatty acid | | |
| composition | | |
| Caprylic acid | <3% | (nominally, 0.2%) |
| (C8) | | |
| Capric acid (C10) | <3% | (nominally, 0.2%) |
| Lauric acid (C12) | <5% | (nominally, 2.2%) |
| Myristic acid | <5% | (nominally, 1.8%) |
| (C14) | | |
| Palmitic acid | 40-50% | (nominally, 42.5%) |
| (C16) | | |
| Stearic acid | 48-58% | (nominally, 52.6%) |
| (C18) | | |

Gelucire ® 53/10

| | | |
|---|---|---|
| Hydroxyl value | 25-45 | mg KOH/g (nominally, 35 mg |
| | | KOH/g) |
| Saponification | 98-112 | mg KOH/g (nominally, 104 mg |
| value | | KOH/g) |
| Fatty acid | | |
| composition | | |
| Caprylic acid | <3% | (nominally, <0.1%) |
| (C8) | | |
| Capric acid (C10) | <3% | (nominally, 0.1%) |
| Lauric acid (C12) | <5% | (nominally, 0.4%) |
| Myristic acid | <5% | (nominally, 1.0%) |
| (C14) | | |
| Palmitic acid | 40-50% | (nominally, 43%) |
| (C16) | | |
| Stearic acid | 48-58% | (nominally, 54.2%) |
| (C18) | | |

The choice in selecting the type(s) of Gelucire® to use in the composition of this invention is based on factors such as desired drug solubilization/loading and release profile. One of the more preferred saturated, polyglycolized glycerides for use in incorporating the taxane derivative in a semisolid matrix for encapsulation is Gelucire 44/14, which provides suitable solubilization of the taxane and immediate/rapid release and dissolution in aqueous media. The use of other grades of Gelucire, or combinations of Gelucire's with different properties, could be utilized to modify the release and dissolution patterns to achieve more sustained delivery of the taxanes with less frequent dosing.

The solid, amphiphilic surfactants used in the practice of this invention are solid at room temperature and are characterized by having hydrophobic and hydrophilic components which impart surface activity to form micelles in which the incorporated drug is protected from macroprecipitation during contact with an aqueous environment such as the GI tract. Preferred solid, amphiphilic surfactants include, without limitation, those selected from the group of hydroxy-substituted stearic acid esters of polyethylene glycol, such as polyethylene glycol 660 12-hydroxystearate (available from BASF Corp., Ludwigshafen, Germany, as Solutol® HS15) and α-tocopheryl-polyethylene succinate esters of polyethylene glycol, also known as PEGylated α-tocopherol derivatives, such as polyethylene glycol-1000-succinate (available from Eastman Chemical Co., Kingsport, Tenn. as TPGS 1000).

Included among the optional components of the solubilizing agent are: an alcohol other than a polyether glycol, such as the monohydric alcohols ethanol, 2-(2-ethoxyethoxy) ethanol (Transcutol®, available from Gattefosse Corp.) and benzylalcohol, as well as the monomeric, polyhydric alcohols propylyene glycol, glycerol and the like; fatty acid ester derivatives of polyhydric alcohols, such as medium chain fatty acid monoglycerides, diglycerides (e.g. Capmul MCM, available from Abitech Corp., Janesville, Wis.), triglycerides and mixtures thereof (e.g. Miglyol® 808, Miglyol® 810, Miglyol® 812, Miglyol® 818 and the like; available from Sasol Chemical Industries—North America, Cranford, N.J.; surfactants other than the aforementioned solid, amphiphilic surfactants, such as those selected from the group of polyoxyethlene castor oil derivatives (e.g. polyoxyethyleneglyceroltriricinoleate or polyoxyl 35 castor oil or Cremophor®EL, polyoxyethyleneglyceroloxystearate or polyethyleneglycol 40 hydrogenated castor oil or Cremophor®RH 40, polyethyleneglycol 60 hydrogenated castor oil or Cremophor®RH 60, and the like; (available from BASF Corp., Ludwigshafen, Germany), polyoxyethlene derivatives of fatty acid partial esters of sorbitan, e.g. polyoxyethylene 20 sorbitan monolaurate or Tween®20, polyoxyethylene 40 sorbitan monopalmitate or Tween®40, polyoxyethylene 60 sorbitan monostearate or Tween®60, polyoxyethylene 80 sorbitan monooleate or Tween®80, and the like, polyoxyalkylene derivatives of propylene glycol which are in the form of block copolymers, e.g. Polaxamer 182LF or Pluronic® F62, Polaxamer 188 or Pluronic® F68, Polaxamer 338 or Pluronic® F108, Polaxamer 407 or Pluronic® F127, and the like (available from BASF Corp., Ludwigshafen, Germany), polyoxyethylene glycol stearates, e.g. PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, PEG-150 distearate and the like, sorbitan fatty acid esters, e.g. sorbitan laurate, sorbitan oleate, sorbitain palmitate, sorbiatan stearate and the like, and lecithin; vegetable oils, for example, soybean oil, olive oil, peanut oil and sunflower oil; and mineral oil.

The pharmaceutical compositions described herein may be prepared in various dosage forms, including both solutions and encapsulated solids or semi-solid forms, as exemplified below. Solutions may be encapsulated as semi-solid or solid matrices in capsules made from various materials including, without limitation, geletin, hydroxypropylmethylcellulose (HPMC), cellulose, methyl cellulose, starch and the like. The capsule materials may be either soft or hard. The resulting dosage forms are pharmaceutically acceptable, chemically and physically stable and provide effective and consistent absorption of the taxane derivative.

The choice of ingredients for the dosage forms is influenced primarily by the solubility of the taxane derivative in the component(s) that make(s) up the solubilizing agent. To avoid precipitation of the taxane derivative at typical long-term storage conditions (e.g., 5° C. to 30° C.), the concentration (or percent loading) of the taxane in various dosage form compositions is preferably kept below the saturation solubility (either at room temperature if dosage form is liquid at room temperature, or at the solution temperatures used to melt solid ingredients for dosage forms that are semi-solids at room temperature). Table 2 presents solubility of Compound Ia in various composition components. In the case of encapsulated dosage units, the strength (mg of drug per capsule) can be controlled by either modifying the concentration of drug in the fill composition, or by holding the drug concentration constant and modifying the amount of composition filled into the capsule. Each dosage unit of the composition of the invention, irrespective of its physical form, typically contains an amount of the orally effective taxane derivative in the range of from about 2 to about 50.0 mg., with a range of about 5.0 to about 25.0 mg being preferred.

TABLE 2
Solubility of Crystalline Compound Ia in Various
Bioavailability Enhancing Agent Components

| Vehicle (Temperature) | Compound Ia Solubility |
|---|---|
| Water (24 ± 3° C.) | ˜0.007 mg/mL |
| Ethanol, USP (24 ± 3° C.) | ˜200 mg/mL |
| Propylene Glycol (24 ± 3° C.) | ˜40 mg/mL |
| Polyethylene Glycol 400 (24 ± 3° C.) | ˜125 mg/mL |
| Polyethylene Glycol 1450 (70° C.) | ˜70 mg/mL |
| 75% Polyethylene Glycol 400/25% | ˜100 mg/mL |
| Tween 80 (24 ± 3° C.) | |
| Gelucire 44/14 (50° C.) | ˜30 mg/mL |
| TPGS 1000 [Vitamin E PEG 1000 | ˜25 mg/mL |
| Succinate] (50° C.) | |
| Solutol HS 15 (50° C.) | ˜80 mg/mL |
| 50% PEG 400/50% Gelucire 44/14 | ˜80 mg/mL |
| (50° C.) | |
| 50% PEG 400/50% TPGS 1000 (50° C.) | ˜80 mg/mL |
| 25% PEG 400/25% PEG 1450/50% | ˜80 mg/mL |
| Gelucire 44/14 (60° C.) | |
| 25% PEG 400/25% PEG 1450/50% TPGS | ˜80 mg/mL |
| 1000 (60° C.) | |
| 25% PEG 400/25% PEG 1450/50% Tween | ˜80 mg/mL |
| 80 (60° C.) | |
| 28% PEG 400/56% PEG 1450/12% Tween | ˜80 mg/mL |
| 80 (60° C.) | |
| 50% PEG 1450/50% Gelucire 44/14 | ˜70 mg/mL |
| (70° C.) | |
| 50-90% PEG 1450/Tween 80 (70° C.) | ˜70 mg/mL |
| 50% PEG 3350/50% Gelucire 44/14 | ˜60 mg/mL |
| (70° C.) | |
| 50-90% PEG 3350/Tween 80 (70° C.) | ˜60 mg/mL |
| 50% PEG 4000/50% Gelucire 44/14 | ˜60 mg/mL |
| (70° C.) | |
| 50-90% PEG 4000/Tween 80 (70° C.) | ˜60 mg/mL |

The taxane derivative is present in the dosage form at about 1 to 20% by weight, preferably about 4 to 10% by weight. In preferred compositions, one or more polyether glycol solubilizer compounds of various average molecular weights (for example PEG 300, PEG 400, PEG 1450, PEG 3350, and the like) is present in the dosage forms at amounts totaling, by weight, of about 10% to about 99%, preferably about 15% to about 60%. In addition to, or in place of the polyethylene glycol, one or more polyglycolized glyceride solubilizer compounds having amphiphilic properties, such as Gelucire® 44/14, Gelucire® 50/13, Gelucire® 53/10, and the like, can be present in the dosage forms at amounts totaling, by weight, about 10% to about 99%, preferably about 15% to about 60%. In addition to, or in place of the polyether glycol and polyglycolized glyceride, one or more solid, amphiphilic surfactant(s), such as Solutol HS 15 (i.e., polyethylene glycol 660 12-hydroxystearate or Polyoxyl-15-hydroxystearate) and/or PEGylated α-tocopherol derivative, such as TPGS 1000 (i.e., vitamin E polyethylene glycol-1000-succinate or Vitamin E PEG 1000 succinate) can be present in the dosage forms at amounts totaling, by weight, about 10% to about 99%, preferably about 15% to about 60%.

The preferred compositions may also include one or more other surfactants, such as the polyoxyethylene castor oil derivatives (for example, polyoxyethyleneglycerol triricinoleate or polyoxyl 35 castor oil or Cremophor®EL, and the like), and/or sorbitan derivatives (for example, polyoxyethylene 80 sorbitan monooleate or Tween®80, and the like) and/or polyoxyethylene-polyoxypropylene glycol block copolymers (for example Polaxamer 182LF or Pluronic® F62, and the like) at amounts totaling about 5-25%.

Compositions embodying the present invention, as will be seen in the examples provided below, substantially increase absorption of the orally effective taxane derivatives of Formula I and II, compared to the taxane derivative itself, and exhibit relatively low interpatient and intrapatient variability in the extent of absorption.

The dosage forms may optionally contain a pharmaceutically acceptable acid for stabilization of the taxane derivative, including inorganic acids and organic mono-, di-, or tri-carboxylic acids. It has been unexpectedly found that the addition of an organic or inorganic acid to the various solution, semi-solid and solid compositions of Compound Ia can markedly increase the stability of the composition both in solution (either as a dosage form or prior to capsule filling) or as a semi-solid or solid formulation. The acid added to the dosage forms for stabilization of the taxane derivative can be any one or combination of pharmaceutically acceptable inorganic acids (for example: hydrochloric acid, and the like) or organic mono-, di-, or tri-carboxylic acids (for example: acetic acid, ascorbic acid, citric acid, methanesulfonic acid, tartaric acid, and the like). Specific examples of pharmaceutically acceptable acids that are suitable for this purpose and amounts of such acids that are effective for increasing the storage stability of Compound Ia are set forth herein below.

Other ingredients that may be present in the pharmaceutical compositions of the invention include, for example, the following:

A pharmaceutically acceptable antioxidant for stabilization of the taxane derivative (e.g., ascorbic acid, BHA, BHT, vitamin E, vitamin E PEG 1000 succinate, and the like).

At least one or more precipitation inhibitor such as the polyvinylpyrrolidinone (PVP or povidone) polymers of various molecular weights (e.g., polyvinylpyrrolidinone K12-18, average MW 10,000, polyvinylpyrrolidinone K30-18, average MW 40,000, and the like); or water-soluble cellulose ether derivatives (e.g., hydroxypropylcellulose, hydroxypropylmethylcellulose, and the like).

Added water to improve the compatibility of the compositions with the hard or soft capsule shell thereby enhancing physical stability. The addition is particularly beneficial in the case of compositions contining polyethylene glycol which, for example, due to their hygroscopic nature (for example polyethylene) tend to extract water from the capsule shell.

Glycerin or another suitable plasticizer for promoting physical stability when encapsulated in a soft gelatin capsule.

Further details regarding the practice of this invention are set forth in the following examples, which are provided for illustrative purposes only and are in no way intended to limit the invention.

### EXAMPLE 1

### Capsule

Compound Ia was added to a batching vessel containing polyethylene glycol 400, pre-melted polyethylene glycol 1450 and pre-melted Gelucire 44/14 and mixed at about 65° C. to dissolve the drug and give a solution at 4% by weight. The solution was filled into size #2, #1 and #0 gray, opaque hard gelatin capsule shells at 50, 125 and 625 mg amounts, respectively, to provide dosage forms at strengths of 2, 5 and 25 mg of the taxane derivative per capsule, respectively. Caps were placed on the filled capsule bodies after they were stored at room temperature for about 30-60 minutes to solidify the filled contents. The recommended storage condition for the capsules is 12 months at controlled room temperature of 15-25° C. (59-77° F.). The dosage forms exhibit high potency recovery, rapid dissolution, and maintain excellent chemical, physical and dissolution stability during long-term storage, including no evidence of drug crystallization in the semi-solid matrix. Dissolution studies in water (in the absence of added surfactant) indicate the semi-solid matrix erodes to a very fine dispersion rather than a macroparticulate suspension. Capsules were administered to cancer patients in Phase I clinical studies to determine various in vivo parameters following oral dosing, such as safety and pharmacokinetic profiles across different dose ranges and schedules, including bioavailability, intra- and inter-patient variability. Absolute oral bioavailability was determined by co-administering a 50 mg dose (i.e., two 25 mg strength capsules) of the capsule formulation orally along with an intravenously administered 25 mg dose of a solution formulation of a ¹³C-labeled form of the drug. The absolute oral bioavailability (F) shown is the mean value from the pharmacokinetic profiles of six patients. Based on comparable in vitro dissolution profiles of the 2 mg and 25 mg strength capsules of each formulation, the absolute oral bioavailability would be anticipated to be equivalent if 2 mg or 5 mg strength capsules were administered to provide the same dose (i.e., 25 2 mg strength capsule or ten 5-mg strength capsules to dose 50 mg total of Compound Ia). The same is true of the value measured for the coefficient of variation (c.v.) for the formulations of this Example 1, which was determined by dividing the mean value for absolute oral bioavailability into the standard deviation, then multiplying by 100 to express as a percentage.

| | Composition | | Composition | | Composition | |
|---|---|---|---|---|---|---|
| | A | | B | | C | |
| | Amount | | Amount | | Amount | |
| | (mg) | | (mg) | | (mg) | |
| | per | % of | per | % of | per | % of |
| Ingredient | Capsule | Total | Capsule | Total | Capsule | Total |
| Compound Ia | 2.0 | 4.0% | 5.0 | 4.0% | 25.0 | 4.0% |
| PEG 400 | 12.0 | 24.0% | 30.0 | 24.0% | 150.0 | 24.0% |
| PEG 1450 | 12.0 | 24.0% | 30.0 | 24.0% | 150.0 | 24.0% |
| Gelucire 44/14 | 24.0 | 48.0% | 60.0 | 48.0% | 300.0 | 48.0% |
| Total | 50.0 | 50.0% | 125.0 | 100.0% | 625.0 | 100.0% |

Pharmacokinetics

| | |
|---|---|
| F (Oral Bioavailability) | 24% |
| C.V. (Coefficient of Variation) | 45% |

### EXAMPLE 2

### Capsule

Compound Ia was added to a batching vessel containing polyethylene glycol 400, Tween®80, and pre-melted polyethylene glycol 1450 and mixed at about 65° C. to dissolve the drug and give a solution at 4% by weight. The solution was filled into size #0 gray, opaque hard gelatin capsules at 625 mg to provide a dosage form at a strength of 25 mg of the taxane derivative per capsule. Caps were placed on the filled capsule bodies after they were stored at room temperature for about 30-60 minutes to solidify the filled contents. The recommended storage condition for the capsules is 12 months at controlled room temperature of 15-25° C. (59-77° F.). The dosage form exhibits high potency recovery, rapid dissolution, and maintains excellent chemical, physical and dissolution stability during long-term storage, including no evidence of drug crystallization in the semi-solid matrix. Dissolution studies in water (in the absence of added surfactant) indicate the semi-solid matrix erodes to a very fine dispersion rather than a macroparticulate suspension. Capsules were administered to cancer patients in Phase I clinical studies to determine various in vivo parameters following oral dosing, such as safety and pharmacokinetic profiles across different dose ranges and schedules, including bioavailability, intra- and inter-patient variability. Absolute oral bioavailability and coefficient of variations were determined as described above in Example 1.

| | | |
|---|---|---|
| | Amount (mg) | |
| Ingredient | per Capsule | Percentage of Total |

Composition D

| | | |
|---|---|---|
| Compound Ia | 25.0 | 4.0% |
| PEG 400 | 175.0 | 28.0% |
| PEG 1450 | 350.0 | 56.0% |
| Tween 80 | 75.0 | 12.0% |
| Total | 625.0 | 100.0% |

Pharmacokinetics

| | |
|---|---|
| F (Oral Bioavailability) | 23% |
| C.V. (Coefficient of Variation) | 30% |

### EXAMPLE 3

### Capsule

Compound Ia was added to a batching vessel containing polyethylene glycol 400, pre-melted polyethylene glycol 1450 and pre-melted Gelucire® 44/14 and mixed at about 65° C. to dissolve the drug and give a solution at 4%. by weight.

The solution was filled into size #1 gray, opaque hard gelatin capsules at 500 mg to provide a dosage form at a strength of 20 mg of the taxane derivative per capsule. Caps were placed on the filled capsule bodies after they were stored at room temperature, for about 30-60 minutes to solidify the filled contents. Capsules were dosed to each of 2 dogs at a dose of approximately 2 mg/kg and plasma samples were taken and analyzed for pharmacokinetic parameters including drug concentrations versus time. Absolute oral bioavailability and coefficient of variation were determined as described above in Example 1.

| | | |
|---|---|---|
| | | Percentage of |
| Ingredient | Amount (mg) | Total |

Composition E

| | | |
|---|---|---|
| Compound Ia | 20.0 | 4.0% |
| PEG 400 | 120.0 | 24.0% |
| PEG 1450 | 120.0 | 24.0% |
| Gelucire 44/14 | 240.0 | 48.0% |
| Total | 500.0 | 100.0% |

Pharmacokinetics

| | |
|---|---|
| F (Oral Bioavailability) | 29% |
| C.V. (Coefficient of Variation) | 19% |

### EXAMPLE 4

### Capsule

Compound Ia was dissolved at 10% by weight in pre-melted Gelucire 44/14 at about 65° C. and the solution was filled into size #1 gray, opaque hard gelatin capsules. Caps were placed on the filled capsule bodies after they were stored at room temperature for about 30-60 minutes to solidify the filled contents. Capsules were dosed to each of 3 dogs at a dose of approximately 3 mg/kg and plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | | |
|---|---|---|
| Ingredient | Amount (mg) | Percentage of Total |

Composition F

| | | |
|---|---|---|
| Compound Ia | 30.0 | 10.0% |
| Gelucire 44/14 | 270.0 | 90.0% |
| Total | 300.0 | 100.0% |

Pharmacokinetics

| | |
|---|---|
| F (Oral Bioavailability) | 32.7% |
| C.V. (Coefficient of Variation) | 2% |

### EXAMPLE 5

### Capsule

Compound Ia was dissolved at 10% by weight in pre-melted Solutol HS 15 at about 65° C. and the solution was filled into size #1 gray, opaque hard gelatin capsules. Caps were placed on the filled capsules after they were stored at room temperature for about 30-60 minutes to solidify the filled contents. Capsules were dosed to each of 3 dogs at a dose of approximately 3 mg/kg and plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | | |
|---|---|---|
| Ingredient | Amount (mg) | Percentage of Total |

Composition G

| | | |
|---|---|---|
| Compound Ia | 30.0 | 10.0% |
| Solutol HS 15 | 270.0 | 90.0% |
| Total | 300.0 | 100.0% |

Pharmacokinetics

| | |
|---|---|
| F (Oral Bioavailability) | 42.8% |
| C.V. (Coefficient of Variation) | 44% |

### EXAMPLE 6

### Capsule

Compound Ia was dissolved at 10% by weight in pre-melted TPGS 1000 (vitamin E PEG 1000 succinate) at about 65° C. and the solution was filled into size #1 gray, opaque hard gelatin capsules. Caps were placed on the filled capsule bodies after they were stored at room temperature for about 30-60 minutes to solidify the filled contents. Capsules were dosed to each of 3 dogs at a dose of approximately 3 mg/kg and plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | | |
|---|---|---|
| Ingredient | Amount (mg) | Percentage of Total |

Composition H

| | | |
|---|---|---|
| Compound Ia | 30.0 | 10.0% |
| TPGS 1000 | 270.0 | 90.0% |
| Total | 300.0 | 100.0% |

Pharmacokinetics

| | |
|---|---|
| F (Oral Bioavailability) | 33.6% |
| C.V. (Coefficient of Variation) | 8% |

### EXAMPLE 7

### Capsule

Compound Ia was dissolved at 4% by weight in a combination of PEG 400 and pre-melted Gelucire® 44/14 at about 65° C. and the solution was filled into size #1 gray, opaque hard gelatin capsules. Caps were placed on the filled capsule bodies after they were stored at room temperature for about 30-60 minutes to solidify the filled contents. Capsules were dosed to each of 3 dogs at a dose of approximately 2 mg/kg and plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | | |
|---|---|---|
| Ingredient | Amount (mg) | Percentage of Total |

Composition I

| | | |
|---|---|---|
| Compound Ia | 20.0 | 4.0% |
| PEG 400 | 240.0 | 48.0% |
| Gelucire 44/14 | 240.0 | 48.0% |
| Total | 500.0 | 100.0% |

Pharmacokinetics

| | |
|---|---|
| F (Oral Bioavailability) | 31.3% |
| C.V. (Coefficient of Variation) | 4% |

### EXAMPLE 8

### Capsule

Compound Ia was dissolved at 4% by weight in a combination of PEG 400 and pre-melted TPGS 1000 (vitamin E PEG 1000 succinate) at about 65° C. and the solution was filled into size #1 gray, opaque hard gelatin capsules. Caps were placed on the filled capsule bodies after they were stored at room temperature for about 30-60 minutes to solidify the filled contents. Capsules were administered to each of 3 dogs at a dose of approximately 2 mg/kg and plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | | |
|---|---|---|
| Ingredient | Amount (mg) | Percentage of Total |

Composition J

| | | |
|---|---|---|
| Compound Ia | 20.0 | 4.0% |
| PEG 400 | 240.0 | 48.0% |
| TPGS 1000 | 240.0 | 48.0% |
| Total | 500.0 | 100.0% |

Pharmacokinetics

| | |
|---|---|
| F (Oral Bioavailability) | 24.3% |
| C.V. (Coefficient of Variation) | 10% |

### EXAMPLE 9

### Solution

Compound Ia was dissolved at 4 mg/mL in 75% PEG 400/25% Tween 80 (cleaned by passage through an ion exchange column) and the solution was administered by oral gavage to each of 3 dogs at a dose of approximately 2 mg/kg. Plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | |
|---|---|
| Ingredient | Amount |

Composition K

| | | |
|---|---|---|
| Compound Ia | 6.0 | mg |
| Tween 80 | 0.25 | mL |
| PEG 400 | q.s. to 1.0 | mL |
| Total | 1.0 | mL |

Pharmacokinetics

| | |
|---|---|
| F (Oral Bioavailability) | 29.3% |
| C.V. (Coefficient of Variation) | 10% |

### EXAMPLE 10

### Solution

Compound Ia was dissolved at 6 mg/mL in PEG 400 and the solution was administered by oral gavage to each of 3 dogs at a dose of approximately 3 mg/kg. Plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | |
|---|---|
| Ingredient | Amount |

Composition L

| | | |
|---|---|---|
| Compound Ia | 6.0 | mg |
| PEG 400 | q.s. to 1.0 | mL |
| Total | 1.0 | mL |

Pharmacokinetic

| | |
|---|---|
| F (Oral | 15.6% |
| Bioavailability) | |
| C.V. (Coefficient of | 45% |
| Variation) | |

### EXAMPLE 11

### Solution

Compound Ia was dissolved at 6 mg/mL in Labrafil M1944CS (an unsaturated polyglycolized glyceride) and the solution was administered by oral gavage to each of 3 dogs at a dose of approximately 3 mg/kg. Plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | |
|---|---|
| Ingredient | Amount |

Composition M

| | | |
|---|---|---|
| Compound Ia | 6.0 | mg |
| Labrafil M1944CS | q.s. to 1.0 | mL |
| Total | 1.0 | mL |

Pharmacokinetics

| | |
|---|---|
| F (Oral | 8.6% |
| Bioavailability) | |
| C.V. (Coefficient of | 27% |
| Variation) | |

### EXAMPLE 12

### Solution

Compound Ia was dissolved at 4 mg/mL in 75% PEG 400/25% Cremophor EL (cleaned by passage through an ion exchange column) and the solution was administered by oral gavage to each of 3 dogs at a dose of approximately 2 mg/kg. Plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | |
|---|---|
| Ingredient | Amount |

Composition N

| | | |
|---|---|---|
| Compound Ia | 6.0 | mg |
| Cremophor EL | 0.25 | mL |
| PEG 400 | q.s. to 1.0 | mL |
| Total | 1.0 | mL |

Pharmacokinetics

| | |
|---|---|
| F (Oral | 7.5% |
| Bioavailability) | |
| C.V. (Coefficient of | 2% |
| Variation) | |

### EXAMPLE 13

### Capsule

Compound II was added to a batching vessel containing pre-melted Gelucire® 44/14 and mixed at about 65° C. to dissolve the drug and give a solution at 20% w/w. The solution was filled into size #1 gray, opaque hard gelatin capsules at 250 mg to provide a dosage form at a strength of 50 mg of Compound II per capsule. Caps were placed on the filled capsules after they were stored at room temperature for about 30-60 minutes to solidify the filled contents. The dosage form maintained rapid and full dissolution and excellent chemical and physical stability during long-term storage at 5 and 25° C.

Composition O

| | Amount | |
|---|---|---|
| | (mg) per | Percentage of |
| Ingredient | Capsule | Total |
| Compound II | 50.0 | 20.0% |
| Gelucire 44/14 | 200.0 | 80.0% |
| Total | 250.0 | 100.0% |

### EXAMPLE 14

### Capsule

Compound II was added to a batching vessel containing pre-melted Gelucire 44/14 and Cremophor EL (cleaned by passage through an ion exchange column) and mixed at about 65° C. to dissolve the drug and give a solution at 20% w/w. The solution was filled into size #1 gray, opaque hard gelatin capsules at 250 mg to provide a dosage form at a strength of 50 mg of Compound II per capsule. Caps were placed on the filled capsules after they were stored at room temperature for about 30-60 minutes to solidify the filled contents. The dosage form maintained rapid and full dissolution and excellent chemical and physical stability during long-term storage at 5 and 25° C.

Composition P

| | Amount | |
|---|---|---|
| | (mg) per | Percentage of |
| Ingredient | Capsule | Total |
| Compound II | 50.0 | 20.0% |
| Gelucire 44/14 | 150.0 | 60.0% |
| Cremophor EL | 50.0 | 20.0% |
| Total | 250.0 | 100.0% |

### EXAMPLE 15

### Capsule

Compound II was added to a batching vessel containing pre-melted Gelucire® 44/14 and pre-melted Solutol HS 15 and mixed at about 65° C. to dissolve the drug and give a solution at 20% w/w. The solution was filled into size #1 gray, opaque hard gelatin capsules at 250 mg to provide a dosage form at a strength of 50 mg of Compound II per capsule. Caps were placed on the filled capsule bodies after they were stored at room temperature for about 30-60 minutes to solidify the filled contents. The dosage form maintained rapid and full dissolution and excellent chemical and physical stability during long-term storage at 5 and 25° C.

Composition Q

| | Amount | |
|---|---|---|
| | (mg) per | Percentage of |
| Ingredient | Capsule | Total |
| Compound II | 50.0 | 20.0% |
| Gelucire 44/14 | 150.0 | 60.0% |
| Solutol HS 15 | 50.0 | 20.0% |
| Total | 250.0 | 100.0% |

### EXAMPLE 16

### Capsule

Compound Ig was added to a batching vessel containing pre-melted Gelucire® 44/14 and mixed at about 65° C. to dissolve the drug and give a solution at 10% w/w. The solution was filled into size #1 gray, opaque hard gelatin capsules at 200 mg to provide a dosage form at a strength of 20 mg of Compound Ig per capsule. Caps were placed on the filled capsules after they were stored at room temperature for about 30-60 minutes to solidify the filled contents. The dosage form displayed rapid and full dissolution.

Composition R

| | Amount | |
|---|---|---|
| | (mg) per | Percentage of |
| Ingredient | Capsule | Total |
| Compound Ig | 20.0 | 10.0% |
| Gelucire 44/14 | 180.0 | 90.0% |
| Total | 200.0 | 100.0% |

### EXAMPLE 17

### Capsule

Compound Ig was added to a batching vessel containing pre-melted PEG 1450 and mixed at about 65° C. to dissolve the drug and give a solution at 10% w/w. The solution was filled into size #1 gray, opaque hard gelatin capsules at 200 mg to provide a dosage form at a strength of 20 mg of Compound Ig per capsule. Caps were placed on the filled capsules after they were stored at room temperature for about 30-60 minutes to solidify the filled contents. The dosage form displayed rapid and full dissolution.

Composition S

| | Amount | |
|---|---|---|
| | (mg) per | Percentage of |
| Ingredient | Capsule | Total |
| Compound Ig | 20.0 | 10.0% |
| PEG 1450 | 180.0 | 90.0% |
| Total | 200.0 | 100.0% |

### EXAMPLE 18

### Capsule

Compound Ig was added to a batching vessel containing pre-melted PEG 3350 and mixed at about 65° C. to dissolve the drug and give a solution at 10% w/w. The solution was filled into size #1 gray, opaque hard gelatin capsules at 200 mg to provide a dosage form at a strength of 20 mg of Compound Ig per capsule. Caps were placed on the filled capsules after they were stored at room temperature for about 30-60 minutes to solidify the filled contents. The dosage form displayed a modified release pattern with a slower dissolution rate to provide for a more sustained delivery of the drug.

Composition T

| | Amount | |
|---|---|---|
| | (mg) per | Percentage of |
| Ingredient | Capsule | Total |
| Compound Ig | 20.0 | 10.0% |
| PEG 3350 | 180.0 | 90.0% |
| Total | 200.0 | 100.0% |

### EXAMPLE 19

### Solution

Compound Ig was dissolved at 8 mg/mL in Labrasol and the solution was administered by oral gavage to each of 5 rats at a dose of approximately 15 mg/kg. Plasma samples were taken and analyzed for pharmacokinetic parameters including drug concentrations versus time. The AUC's were calculated and used to determine the absolute oral bioavailability relative to Compound Ig administered intravenously to rats from a cremophor/ethanol/water solution.

| | |
|---|---|
| Ingredient | Amount |

Composition U

| | | |
|---|---|---|
| Compound Ig | 8.0 | mg |
| Labrasol | q.s. to 1.0 | mL |
| Total | 1.0 | mL |

Pharmacokinetics

| | |
|---|---|
| F (Oral | 14.1% |
| Bioavailability) | |
| C.V. (Coefficient of | 7.3% |
| Variation) | |

Acid-stabilized dosage forms of the present invention are described in the following examples:

### EXAMPLE 20

Capsule formulations comprising Compound Ia, a solubulizing agent and an effective amount of a pharmaceutically acceptable acid stabilizer were prepared according to the following general procedure:

1. Add weighed amount of selected solubulizing agent component(s) (in liquid, powder, granular or pre-melted molten form) to a batching vessel pre-equilibrated to approximately 70° C.

2. Begin stirring to completely melt any solid component(s) of the solubulizing agent at approximately 70° C. to obtain a clear, homogeneous solution.

3. Add weighed amount of stabilizer acid to the stirring solubulizing agent from step 2 and continue stirring at 70° C.

4. Continue stirring at approximately 70° C. to completely mix and dissolve the acid stabilizer.

5. Slowly add weighed amount of Compound Ia to the stirring mixture of solubilizing agent and acid stabilizer from step 4 with continuous stirring at 70° C.

6. Continue stirring the mixture from step 5 at approximately 70° C. to give a clear, homogeneous solution.

7. Fill an appropriate amount of the solution from step 6 into capsule shells to provide capsules of various dosage strengths. For formulation solutions having a taxane derivative content of 4 wt %, for example, 5 mg strength and 25 mg strength capsules are prepared by filling 125 mg and 625 mg of the formulation solutions into Size #1 (or #2) and Size #0 two-piece hard gelatin capsule shells, respectively.

8. Allow the contents of the capsules from step 7 to solidify.

9. Place the caps on the filled capsule bodies from step 8.

The Compound Ia potency and impurity/degradation product profile were evaluated and compared using the HPLC assay methodology described immediately below.

1. The cap is removed from one or more capsules and the capsule(s) containing the semi-solid formulation contents are placed in a glass volumetric flask. Acetonitrile is added to bring the flask to exact volume. Typically, the number of capsules and volume of acetonitrile added are selected to achieve a final taxane derivative concentration of 0.25 mg/mL (e.g., one 25-mg strength capsule or five 5-mg strength capsules in a 100 mL volumetric flask, etc.).

2. The flask is sealed, placed in an ultrasonic bath, and the sample is sonicated for approximately 30 minutes, with periodic shaking of the flask, to completely dissolve and mix the formulation contents into the acetonitrile.

3. An aliquot of the solution is then assayed using the following gradient HPLC assay methodology: A 20 microliter aliquot of the sample is injected onto a C18 reverse-phase HPLC column (YMC ODS-AQ, 150 mm length×4.6 mm i.d., 3 μm particle size, 120A pore volume) and eluted using a gradient mobile phase system (shown below) at a solvent flow rate of 1.0 mL/minute for a 70 minute run time. During elution, the solution is continually exposed to ultraviolet light at a wavelength of 240 nm to detect the parent taxane derivative peak and associated impurity/degradant peaks. The signal generated from the absorbance of ultraviolet light by the component(s) present in the sample is converted from analog to digital and expressed as a peak in the chromatogram baseline signal monitored throughout the elution run time. The peak area is integrated using chromatography peak integration software. The amount of parent taxane derivative present in the sample (typical peak retention time approximately 33 minutes) is quantified by comparing the peak area of the sample with that of a standard solution of drug prepared at known concentration. The amount of impurity/degradant present is reported as I.I. (impurity index), which is an estimate of the amount of an impurity/degradant present in a sample and is calculated from the ratio of the peak area of the impurity/degradant relative to the total peak area of all the sample components normalized by multiplying this ratio by 100. The I.I. is determined when the component is measured without comparison to standard and without correcting the peak area of the impurity/degradant for the relative response factor. The identity of unknown impurities/degradants is typically reported as their respective HPLC retention time in minutes, or by their HPLC relative retention time (RRT, no units) which is the retention time of the impurity/degradant peak relative to the retention time of the parent peak.

Gradient Elution Program

| | Percent | Percent | |
|---|---|---|---|
| Time (minutes) | Acetonitrile | Water | Gradient Profile |
| 0 | 45 | 55 | Isocratic |
| 4 | 45 | 55 | Isocratic |
| 14 | 52 | 48 | Linear |
| 39 | 52 | 48 | Isocratic |
| 59 | 90 | 10 | Linear |
| 62 | 90 | 10 | Isocratic |
| 65 | 45 | 55 | Linear |
| 70 | 45 | 55 | Isocratic |

Table 3, below, shows the beneficial effect of ids on the stabilization of Compound Ia-containing dosage formulations prepared according to the described immediately above after seven (7) days at 70° C., as compared to formulations having no added acid. The formulations were prepared as a solution composed of 3 weight % Compound Ia; 84.9 weight % polyethylene glycol 1450; 12 weight % Tween® 80.

TABLE 3
Impurity/Degradant Level (Peak Area %)

| | | | | Total |
|---|---|---|---|---|
| | Degradant | Degradant | Degradant | Impurity/ |
| Acid | #1 | #2 | #3 | Degradants |
| No Acid | 0.32 | 2.01 | 0.65 | 4.3 |
| 0.1% Acetic | Not Detected | 1.91 | 0.51 | 3.1 |
| Acid | | | | |
| 0.1% Benzoic | Not Detected | 2.18 | 0.38 | 3.6 |
| Acid | | | | |
| 0.1% Citric | 0.07 | 0.71 | 0.14 | 1.6 |
| Acid | | | | |
| 0.1% Maleic | 0.12 | 2.24 | 0.31 | 3.3 |
| Acid | | | | |
| 0.1% Phosphoric | 0.55 | 0.16 | 0.89 | 2.5 |
| Acid | | | | |
| 0.1% Succinic | 0.17 | 1.65 | 0.35 | 2.7 |
| Acid | | | | |
| 0.1% Tartaric | 0.50 | 0.30 | 0.18 | 1.9 |
| Acid | | | | |

In Table 4, below, it can be seen that the beneficial effect obtained from the addition of citric acid to the basic formulation of Table 3 is maintained over a broad concentration of added acid. This stability testing was performed after maintaining the solution, prepared and encapsulated as described immediately above, for a period of from one (1) to seven (7) days at 70° C.

TABLE 4
Impurity/Degradant Level (Peak Area %)

| | | | | | Total |
|---|---|---|---|---|---|
| | | Degradant | Degradant | Degradant | Impurity/ |
| | | #1 | #2 | #3 | Degradants |
| No Acid | 1 Day 70° C. | 0.18 | 0.31 | 0.30 | 1.1 |
| | 3 Day 70° C. | 0.26 | 0.71 | 0.44 | 2.0 |
| | 7 Day 70° C. | 0.32 | 2.01 | 0.65 | 4.3 |
| 0.1% Citric | 1 Day 70° C. | Not | 0.18 | 0.10 | 0.6 |
| | | Detected | | | |
| Acid | 3 Day 70° C. | 0.16 | 0.37 | 0.12 | 1.0 |
| | 7 Day 70° C. | 0.07 | 0.71 | 0.14 | 1.6 |
| 1.0% Citric | 1 Day 70° C. | 0.05 | 0.11 | 0.10 | 0.6 |
| Acid | 3 Day 70° C. | 0.11 | 0.33 | 0.16 | 0.9 |

As is evident from the data presented in Table 5, below, the addition of citric acid is effective for stabilizing various of the enhanced bioavailability dosage formulations of orally-active taxane derivatives embodying the present invention. The formulations were prepared as solutions containing 3 weight % of Compound Ia and 96.9 weight % of a solubilizing agent, with or without optional surfactant, and 0.1 weight % of citric acid. The solutions were prepared and encapsulated as described immediately above. The stability testing was performed after maintaining the solution for seven (7) days at 70° C.

TABLE 5
Impurity/Degradant Level (Peak Area %)

| | | | | Total |
|---|---|---|---|---|
| | Degradant | Degradant | Degradant | Impurity/ |
| | #1 | #2 | #3 | Degradants |
| 3% Compound Ia/85.0% PEG 1450/12.0% | 0.32 | 2.01 | 0.65 | 4.3 |
| Tween 80/No Acid | | | | |
| 3% Compound Ia/72.9% PEG 1450/24.0% | 0.09 | 0.81 | 0.12 | 1.7 |
| Tween 80/0.1% Citric Acid | | | | |
| 3% Compound Ia/84.9% PEG 1450/12.0% | 0.11 | 0.84 | 0.20 | 1.6 |
| Tween 80/0.1% Citric Acid | | | | |
| 3% Compound Ia/72.9% PEG 3350/24.0% | 0.20 | 0.87 | 0.12 | 1.9 |
| Tween 80/0.1% Citric Acid | | | | |
| 3% Compound Ia/84.9% PEG 3350/12.0% | 0.15 | 0.71 | 0.17 | 1.8 |
| Tween 80/0.1% Citric Acid | | | | |
| 3% Compound Ia/72.9% PEG 4000/24.0% | 0.14 | 1.27 | 0.13 | 2.1 |
| Tween 80/0.1% Citric Acid | | | | |
| 3% Compound Ia/84.9% PEG 4000/12.0% | 0.21 | 1.28 | 0.17 | 2.2 |
| Tween 80/0.1% Citric Acid | | | | |
| 3% Compound Ia/48.4% PEG 1450/48.4% | 0.17 | 0.38 | 0.23 | 1.7 |
| Gelucire 44/14/0.1% Citric Acid | | | | |
| 3% Compound Ia/48.4% PEG 3350/48.4% | 0.16 | 0.39 | 0.10 | 1.2 |
| Gelucire 44/14/0.1% Citric Acid | | | | |
| 3% Compound Ia/48.4% PEG 4000/48.4% | 0.17 | 0.48 | 0.20 | 1.5 |
| Gelucire 44/14/0.1% Citric Acid | | | | |

The data presented in Table 6 demonstrate that the stability of Compound Ia in dosage formulations containing solubulizer compounds, such as polyethylene glycols, surfactants or the like which have residual levels of alkyl metals, is substantially enhanced by the addition of an acid stabilizer. The dosage formulation solution, which contains 3 weight % of Compound Ia and varying amounts of the solubulizing agent, were prepared and encapsulated as described immediately above. The stability tests on these formulations were conducted after three (3) days at 70° C. (Table 6-1) and after seven (7) days at 70° C. (Table 6-2). Good results were obtained with a citric acid addition of 0.1 wt %.

TABLE 6-1
Impurity/Degradant Level (Peak Area %)

| | | | | | Total |
|---|---|---|---|---|---|
| | Degradant | Degradant | Degradant | Degradant | Impurity/ |
| | #1 | #2 | #3 | #4 | Degradants |
| 3% Compound Ia/85.0% PEG 1450^{a}/12.0% | 8.10 | 15.5 | 36.1 | 24.2 | 93.5 |
| Tween 80^{b}/No Acid | | | | | |
| 3% Compound Ia/84.9% PEG 1450^{a}/12.0% | 0.35 | — | 3.20 | 0.80 | 4.8 |
| Tween 80^{b}/0.1% Citric Acid | | | | | |
| 3% Compound Ia/85.0% PEG 1450^{c}/12.0% | 0.46 | — | 2.30 | 3.80 | 7.0 |
| Tween 80^{d}/No Acid | | | | | |
| 3% Compound Ia/84.9% PEG 1450^{c}/12.0% | 0.13 | — | 0.49 | 0.14 | 1.1 |
| Tween 80^{d}/Citric Acid | | | | | |

^{a}BASF PEG 1450 Lot WPEU-582B (Contains 297 ppm Potassium)
^{b}BMS Tween 80 Lot 9K18029 (Contains <25 ppm Sodium, Potassium)
^{c}Union Carbide PEG 1450 Lot 270403 (Contains 103 ppm Sodium, <25 ppm Potassium)
^{d}J. T. Baker Tween 80 Lot T11594 (Contains 103 ppm Sodium)

TABLE 6-2
Impurity/Degradant Level (Peak Area %)

| | | | | | Total |
|---|---|---|---|---|---|
| | Degradant | Degradant | Degradant | Degradant | Impurity/ |
| | #1 | #2 | #3 | #4 | Degradants |
| 3% Compound Ia/84.90% PEG 3350^{e}/12.00% | — | 0.23 | 0.96 | 0.40 | 2.1 |
| Tween 80^{c}/0.1% Citric Acid | | | | | |
| 3% Compound Ia/84.90% PEG 3350^{e}/12.00% | — | 0.12 | 0.90 | 0.08 | 1.6 |
| Tween 80^{c}/0.5% Citric Acid | | | | | |
| 3% Compound Ia/48.40% PEG 3350^{e}/48.40% | — | 0.16 | 0.32 | 0.11 | 1.5 |
| Gelucire 44/14/0.1% Citric Acid | | | | | |
| 3% Compound Ia/48.25% PEG 3350^{e}/48.25% | — | — | 0.51 | 0.10 | 1.7 |
| Gelucire 44/14/0.5% Citric Acid | | | | | |

^{c}Union Carbide PEG 1450 Lot 270403 (Contains 103 ppm Sodium, <25 ppm Potassium)
^{e}Union Carbide PEG 3350 Lot 170854 (Contains 390 ppm Sodium)

### EXAMPLE 21

Comparative testing was conducted to evaluate the effect of citric acid on stability (i.e, degradation product levels) of certain preferred dosage formulations at the initial timepoint, as determined by characterizing the degradation product profile using the gradient HPLC assay methodology, described above. The formulations tested contained 4 wt % of Compound Ia, solubilizing agents of varying composition, and either 0.1 wt % of citric acid of no added citric acid, as a basis of comparison. The formulations were prepared according to the general procedure described in Example 20, and filled into #0 capsules. As shown in Table 7, at the initial timepoint, the formulations containing 0.1% citric acid display higher Compound Ia potency (i.e., area percent of the peak with relative retention time of 1.00), and much lower levels of degradation products, particularly at RRTs 0.18/0.19, 0.30-0.33, 0.39/0.40, 0.66 and 1.42-1.52) compared to counterpart formulations without citric acid. Furthermore, after 15 months storage at 25° C., the formulations containing 0.1% citric acid continue to display higher Compound Ia potency (i.e., area percent of the peak with relative retention time of 1.00), and lower total levels of degradation products compared to counterpart formulations without citric acid at the initial timepoint. All of the empty spaces in the table indicate the degradant was not formed, or was below the limit of detection (i.e., about 0.05 peak area percent).

TABLE 7
Effect of Citric Acid on Chemical Stability of Compound Ia Capsule Formulations Impurity/Degradant
Index, I.I. (Peak Area Percent at Each Relative Retention Time)

| | | | | | | | | | | | | | | | | |
|---|---|---|---|---|---|---|---|---|---|---|---|---|---|---|---|---|
| | 0.13/ | | 0.18/ | 0.30- | 0.39/ | 0.42/ | | 0.58/ | | 0.78- | | 0.93/ | | 1.05/ | | 1.42- |
| Example No. | 0.14 | 0.17 | 0.19 | 0.33 | 0.40 | 0.44 | 0.47 | 0.60 | 0.66 | 0.80 | 0.89 | 0.94 | 1.00^{a} | 1.06 | 1.30 | 1.52 |

1: 72% PEG 1450/24% Tween 80

| | | | | | | | |
|---|---|---|---|---|---|---|---|
| Initial | 0.10 | 0.33 | 0.32 | 0.28 | 0.08 | 98.4 | 0.08 |

1a: 71.9% PEG 1450/24.0% Tween 80/0.1% Citric Acid

| | | | | | | | | |
|---|---|---|---|---|---|---|---|---|
| Initial | 0.12 | | 0.05 | 0.17 | | | 99.5 | |
| 15 Month 25° C. | 0.06 | 0.06 | 0.04 | | 0.31 | 0.34 | 99.0 | 0.08 |

2: 84% PEG 1450/12% Tween 80

| | | | | | | | |
|---|---|---|---|---|---|---|---|
| Initial | 0.18 | 0.31 | 0.28 | 0.28 | 0.12 | 96.9 | 0.11 |

2a: 83.9% PEG 1450/12.0% Tween 80/0.1% Citric Acid

| | | | | | | |
|---|---|---|---|---|---|---|
| Initial | 0.13 | | 0.18 | | | 99.5 |
| 15 Month 25° C. | 0.06 | 0.06 | | 0.20 | 0.40 | 99.2 |

3: 72% PEG 3350/24% Tween 80 - 25 mg BMS-275183 per Size #0 Capsule

| | | | | | | | | |
|---|---|---|---|---|---|---|---|---|
| Initial | 0.06 | 0.62 | 0.34 | 0.26 | 2.35 | 0.10 | 88.2 | 7.6 |

3a: 71.9% PEG 3350/24.0% Tween 80/0.1% Citric Acid

| | | | | | | | |
|---|---|---|---|---|---|---|---|
| Initial | 0.12 | | 0.06 | 0.13 | | | 99.5 |
| 15 Month 25° C. | 0.06 | 0.06 | | | 0.20 | 0.47 | 99.2 |

4: 84% PEG 3350/12% Tween 80

| | | | | | | | |
|---|---|---|---|---|---|---|---|
| Initial | 1.11 | 0.41 | 0.23 | 3.94 | 0.08 | 76.4 | 17.4 |

4a: 83.9% PEG 3350/12.0% Tween 80/0.1% Citric Acid

| | | | | | | | | |
|---|---|---|---|---|---|---|---|---|
| Initial | 0.13 | | 0.05 | 0.15 | | | 99.5 | |
| 15 Month 25° C. | 0.07 | 0.08 | | | 0.27 | 0.56 | 99.0 | 0.04 |

5: 72% PEG 4000/24% Tween 80

| | | | | | | | | |
|---|---|---|---|---|---|---|---|---|
| Initial | 0.08 | 0.66 | 0.40 | 0.34 | 2.81 | 0.08 | 87.3 | 8.0 |

5a: 71.9% PEG 4000/24.0% Tween 80/0.1% Citric Acid

| | | | | | | | | |
|---|---|---|---|---|---|---|---|---|
| Initial | 0.10 | | 0.05 | 0.12 | 0.06 | | | 99.5 |
| 15 Month 25° C. | 0.07 | 0.11 | | 0.05 | | 0.31 | 0.56 | 98.8 |

6: 84% PEG 4000/12% Tween 80

| | | | | | | | | |
|---|---|---|---|---|---|---|---|---|
| Initial | 0.07 | 10.6 | 0.39 | 0.23 | 4.80 | 0.05 | 74.7 | 18.4 |

6a: 83.9% PEG 4000/12.0% Tween 80/0.1% Citric Acid

| | | | | | | | | | |
|---|---|---|---|---|---|---|---|---|---|
| Initial | 0.11 | | 0.05 | 0.15 | 0.05 | | | 99.5 | 0.05 |
| 15 Month 25° C. | 0.06 | 0.14 | | 0.19 | | 0.57 | 0.82 | 97.9 | 0.16 |

7: 48% PEG 1450/48% Gelucire 44/14

| | | | | | | | | |
|---|---|---|---|---|---|---|---|---|
| Initial | 0.08 | 0.29 | 0.30 | 0.31 | 0.44 | 0.13 | 98.1 | 0.34 |

7a: 47.95% PEG 1450/47.95% Gelucire 44/14/0.1% Citric Acid

| | | | | | | | |
|---|---|---|---|---|---|---|---|
| Initial | 0.13 | | 0.08 | 0.15 | | | 99.5 |
| 15 Month 25° C. | | 0.15 | | | 0.84 | 0.46 | 98.3 |

8: 48% PEG 3350/48% Gelucire 44/14

| | | | | | | | | |
|---|---|---|---|---|---|---|---|---|
| Initial | 0.09 | 0.58 | 0.30 | 0.30 | 1.88 | 0.13 | 93.7 | 2.95 |

8a: 47.95% PEG 3350/47.95% Gelucire 44/14/0.1% Citric Acid

| | | | | | | | | |
|---|---|---|---|---|---|---|---|---|
| Initial | 0.14 | | 0.08 | 0.12 | | | 99.5 | |
| 15 Month 25° C. | 0.08 | 0.20 | | | 0.74 | 0.44 | 98.5 | 0.06 |

9: 48% PEG 4000/48% Gelucire 44/14

| | | | | | | | | |
|---|---|---|---|---|---|---|---|---|
| Initial | 0.08 | 0.61 | 0.30 | 0.26 | 2.16 | 0.11 | 92.9 | 3.43 |

9a: 47.95% PEG 4000/47.95% Gelucire 44/14/0.1% Citric Acid

| | | | | | | | | |
|---|---|---|---|---|---|---|---|---|
| Initial | 0.14 | | 0.08 | 0.15 | | | 99.4 | |
| 15 Month 25° C. | 0.08 | 0.15 | | | 0.62 | 0.43 | 98.6 | 0.06 |

^{a}Compound Ia

### EXAMPLE 22

Dosage formulations in accordance with this invention were prepared following the general procedure described in Example 20, using solubulizing agents composed of PEG 1450 from two different commercial sources (CS No. 1 and CS No. 2) to evaluate possible differences in formulation stability due to the influence of components of the solubulizing agent.

As shown in Table 8, dosage form solutions of Compound Ia in a PEG 400/PEG 1450/Tween®80 composition including PEG 1450 from the two different commercial sources displayed marked differences in stability.

TABLE 8
Impurity/Degradant Index, I.I. (Peak Area Percent at Each Relative Retention Time)

| | | | | | | | | | | | | | |
|---|---|---|---|---|---|---|---|---|---|---|---|---|---|
| 0.13- | | 0.18- | 0.30- | 0.39- | 0.42- | | 0.58- | | | 0.93- | | | 1.42- |
| 0.14 | 0.17 | 0.19 | 0.32 | 0.40 | 0.44 | 0.47 | 0.60 | 0.66 | 0.89 | 0.94 | 1.00 | 1.30 | 1.52 |

Batch A: PEG 1450; CS No. 1 Lot WPEU-582B Granular

| | | | | | | | | | | | |
|---|---|---|---|---|---|---|---|---|---|---|---|
| 24 Hour 65° C. | 0.06 | 4.52 | 1.15 | 0.47 | 0.17 | 0.15 | 0.61 | 6.36 | 0.71 | 58.1 | 26.85 |

Batch B: PEG 1450; CS No. 1 Lot WPHU-596C Granular

| | | | | | | | | | | | | |
|---|---|---|---|---|---|---|---|---|---|---|---|---|
| 24 Hour 65° C. | 0.06 | 0.20 | 1.33 | 0.51 | 0.42 | 0.04 | 0.03 | 0.37 | 2.09 | 0.03 | 83.7 | 11.13 |

Batch C: PEG 1450; CS No. 1 Lot WPYV-502A Molten

| | | | | | | | | | | | |
|---|---|---|---|---|---|---|---|---|---|---|---|
| 24 Hour 65° C. | 0.04 | 2.41 | 0.80 | 0.45 | 0.10 | 0.08 | 0.63 | 4.02 | 68.3 | 0.05 | 22.73 |

Batch D: PEG 1450; CS No. 2 Lot IS793680 Molten

| | | | | | | | | | | | | |
|---|---|---|---|---|---|---|---|---|---|---|---|---|
| 24 Hour 65° C. | 0.07 | 0.06 | 0.07 | 0.52 | 0.47 | 0.03 | 0.05 | 0.31 | 0.10 | 98.0 | 0.09 | 0.05 |

Batch E: PEG 1450; CS No. 2 Lot 270403 Granular

| | | | | | | | | | | | |
|---|---|---|---|---|---|---|---|---|---|---|---|
| 24 Hour 65° C. | 0.07 | 0.06 | 0.17 | 0.53 | 0.46 | 0.09 | 0.19 | 0.13 | 98.1 | 0.10 | 0.11 |
| Control^{a} | 0.07 | 0.05 | 0.15 | 0.58 | 0.47 | | 0.07 | 0.18 | 98.2 | 0.19 | |

^{a}Standard solution of taxane derivative dissolved in acetonitrile at a concentration of about 0.25 mg/mL

The data in Table 8 indicate that the dosage formulations prepared using various batches of PEG 1450 from CS No. 1 consistently exhibited high potency loss of Compound Ia, and formation of significant amounts of various degradants (e.g., at relative HPLC retention times of 0.18-0.19, 0.30-0.32, 0.58-0.60, 0.93-0.94 and 1.42-1.52 minutes) compared to counterpart compound Ia-containing formulations prepared with PEG 1450 from CS No. 2 or the control standard solution of starting drug.

The data in Table 9, by contrast, show that the stability of Compound Ia was dramatically improved when even trace amounts of citric acid were added to the formulation prepared using one of the same batches of PEG 1450 (from CS No. 1) that previously caused significant degradation of the taxane derivative in the absence of the added acid. The formulation under evaluation was composed of the following components, by weight: 4% of Compound Ia, 28% PEG 400, 56% PEG 1450 and 12% Tween® 80. The relative amounts of citric acid added are given in Table 9.

TABLE 9
Impurity/Degradant Index, I.I. (Peak Area Percent at Each Relative Retention Time)

| | | | | | | | | | | |
|---|---|---|---|---|---|---|---|---|---|---|
| 0.13- | 0.18- | 0.30- | 0.39- | 0.58- | | | | | | 1.42- |
| 0.14 | 0.19 | 0.32 | 0.40 | 0.60 | 0.66 | 0.89 | 1.00 | 1.30 | 1.39 | 1.52 |

Batch A-1: PEG 1450; CS No. 1 Lot WPEU-582B Granular ^{a}

| | | | | | | | |
|---|---|---|---|---|---|---|---|
| 24 Hour 65° C. | 2.93 | 0.70 | 0.33 | 0.62 | 5.56 | 73.71 | 15.30 |

Batch B-1: PEG 1450; CS No. 1 Lot WPEU-582B Granular + 0.5% Citric Acid

| | | | | | | | | | |
|---|---|---|---|---|---|---|---|---|---|
| 24 Hour 65° C. | 0.12 | 0.53 | 0.47 | 0.05 | 0.17 | 0.15 | 98.2 | 0.11 | 0.05 |

Batch C-1: PEG 1450; CS No. 1 Lot WPEU-582B Granular + 1.0% Citric Acid

| | | | | | | | | | | |
|---|---|---|---|---|---|---|---|---|---|---|
| 24 Hour 65° C. | 0.15 | 0.53 | 0.46 | 0.08 | 0.15 | 0.16 | 98.3 | 0.10 | 0.02 | 0.01 |

Batch D-1: PEG 1450; CS No. 1 Lot WPEU-582B Granular + 2.0% Citric Acid

| | | | | | | | | | | |
|---|---|---|---|---|---|---|---|---|---|---|
| 24 Hour 65° C. | 0.12 | 0.0.53 | 0.46 | 0.12 | 0.14 | 0.17 | 98.3 | 0.09 | 0.02 | 0.01 |

Batch E-1: PEG 1450; CS No. 1 Lot WPEU-582B Granular + 5.0% Citric Acid

| | | | | | | | | | | |
|---|---|---|---|---|---|---|---|---|---|---|
| 24 Hour 65° C. | | 0.14 | 0.49 | 0.49 | 0.22 | 0.12 | 0.18 | 98.0 | 0.09 | 0.03 |
| Control^{b} | 0.07 | 0.18 | 0.56 | 0.45 | | 0.07 | 0.21 | 98.3 | 0.14 | |

^{a} No added acid
^{b}Standard solution of taxane derivative dissolved in acetonitrile at a concentration of about 0.25 mg/mL.

Additional representative acid-stabilized dosage formulations in accordance with this invention are set forth in the following tables, in which Table 10 lists capsule formulations of Compound Ia at 25 mg strength (4 wt. % drug load); Table 11 lists capsule formulations of Compound Ia at 5 mg strength (4 wt. % drug load); Table 12 lists capsule formulations of Compound Ia at 20 mg strength (3 wt. % drug load); and Table 13 lists capsule formulations of Compound Ia at 5 mg strength (3 wt. % drug load). These capsule formulations, which also contain from 0.1 to 0.5 wt. % citric acid, were prepared in essentially the same manner as described immediately above.

TABLE 10

| | Composition | | | | | |
|---|---|---|---|---|---|---|
| | Compound | Solubilizing | Solubilizing | Citric | | Capsule |
| Formulation | Ia | Agent | Agent | Acid | Total | Size |
| | | PEG 1450 | Gelucire | | | |
| | | | 44/14 | | | |
| 10-1 | 25 mg | 298.4 mg | 298.4 mg | 3.125 mg | 625 mg | Size #0 |
| | (4%) | (47.75%) | (47.75%) | (0.5%) | (100%) | |
| | | PEG 1450 | Tween 80 | | | |
| 10-2 | 25 mg | 521.9 mg | 75 mg | 3.125 mg | 625 mg | Size #0 |
| | (4%) | (83.5%) | (12.0%) | (0.5%) | (100%) | |
| | | PEG 3350 | Gelucire | | | |
| | | | 44/14 | | | |
| 10-3 | 25 mg | 299.0 mg | 299.0 mg | 1.875 mg | 625 mg | Size #0 |
| | (4%) | (47.85%)^{a} | (47.85%) | (0.3%) | (100%) | |
| 10-4 | 25 mg | 298.4 mg | 298.4 mg | 3.125 mg | 625 mg | Size #0 |
| | (4%) | (47.75%)^{a} | (47.75%) | (0.5%) | (100%) | |
| | | PEG 3350 | Tween 80 | | | |
| 10-5 | 25 mg | 523.1 mg | 75 mg | 1.875 mg | 625 mg | Size #0 |
| | (4%) | (83.7%) ^{a} | (12.0%) | (0.3%) | (100%) | |
| 10-6 | 25 mg | 521.9 mg | 75 mg | 3.125 mg | 625 mg | Size #0 |
| | (4%) | (83.5%)^{a} | (12.0%) | (0.5%) | (100%) | |
| | | PEG 4000 | Gelucire | | | |
| | | | 44/14 | | | |
| 10-7 | 25 mg | 298.4 mg | 298.4 mg | 3.125 mg | 625 mg | Size #0 |
| | (4%) | (47.75%) | (47.75%) | (0.5%) | (100%) | |
| | | PEG 4000 | Tween 80 | | | |
| 10-8 | 25 mg | 521.9 mg | 75 mg | 3.125 mg | 625 mg | Size #0 |
| | (4%) | (83.5%) | (12.0%) | (0.5%) | (100%) | |

^{a}PEG 3350 with high residual alkali (390 ppm sodium)

TABLE 11

| | Composition | | | | | |
|---|---|---|---|---|---|---|
| | Compound | Solubilizing | Solubilizing | Citric | | Capsule |
| Formulation | Ia | Agent | Agent | Acid | Total | Size |
| | | PEG 1450 | Gelucire | | | |
| | | | 44/14 | | | |
| 11-1 | 5 mg | 59.9 mg | 59.9 | 0.125 mg | 125 mg | Size #2 |
| | (3%) | (47.95%) | (47.95%) | (0.1%) | (100%) | |
| 11-2 | 5 mg | 59.7 mg | 59.7 mg | 0.625 mg | 125 mg | Size #2 |
| | (3%) | (47.75%) | (47.75%) | (0.5%) | (100%) | |
| | | PEG 1450 | Tween 80 | | | |
| 11-3 | 5 mg | 89.9 mg | 30 mg | 0.125 mg | 125 mg | Size #2 |
| | (3%) | (71.9%) | (24.0%) | (0.1%) | (100%) | |
| 11-4 | 5 mg | 104.9 mg | 15 mg | 0.125 mg | 125 mg | Size #2 |
| | (3%) | (83.9%) | (12.0%) | (0.1%) | (100%) | |
| 11-5 | 5 mg | 104.4 mg | 15 mg | 0.625 mg | 125 mg | Size #2 |
| | (3%) | (83.5%) | (12.0%) | (0.5%) | (100%) | |
| | | PEG 3350 | Gelucire | | | |
| | | | 44/14 | | | |
| 11-6 | 5 mg | 59.9 mg | 59.9 mg | 0.125 mg | 125 mg | Size #2 |
| | (3%) | (47.95%)^{a,b} | (47.95%) | (0.1%) | (100%) | |
| 11-7 | 5 mg | 59.8 mg | 59.8 mg | 0.375 mg | 125 mg | Size #2 |
| | (3%) | (47.85%)^{a} | (47.85%) | (0.3%) | (100%) | |
| 11-8 | 5 mg | 59.7 mg | 59.7 mg | 0.625 mg | 125 mg | Size #2 |
| | (3%) | (47.75%)^{a} | (47.75%) | (0.5%) | (100%) | |
| | | PEG 3350 | Tween 80 | | | |
| 11-9 | 5 mg | 89.9 mg | 30 mg | 0.125 mg | 125 mg | Size #2 |
| | (3%) | (71.9%)^{b} | (24.0%) | (0.1%) | (100%) | |
| 11-10 | 5 mg | 104.9 mg | 15 mg | 0.125 mg | 125 mg | Size #2 |
| | (3%) | (83.9%)^{a,b} | (12.0%) | (0.1%) | (100%) | |
| 11-11 | 5 mg | 104.6 mg | 15 mg | 0.375 mg | 125 mg | Size #2 |
| | (3%) | (83.7%)^{a} | (12.0%) | (0.3%) | (100%) | |
| 11-12 | 5 mg | 104.4 mg | 15 mg | 0.625 mg | 125 mg | Size #2 |
| | (3%) | (83.5%)^{c} | (12.0%) | (0.5%) | (100%) | |
| | | PEG 4000 | Gelucire | | | |
| | | | 44/14 | | | |
| 11-13 | 5 mg | 59.9 mg | 59.9 mg | 0.125 mg | 125 mg | Size #2 |
| | (3%) | (47.95%)^{c} | (47.95%) | (0.1%) | (100%) | |
| 11-14 | 5 mg | 59.7 mg | 59.7 mg | 0.625 mg | 125 mg | Size #2 |
| | (3%) | (47.75%) | (47.75%) | (0.5%) | (100%) | |
| | | PEG 4000 | Tween 80 | | | |
| 11-15 | 5 mg | 89.9 mg | 30 mg | 0.125 mg | 125 mg | Size #2 |
| | (3%) | (71.9%)^{c} | (24.0%) | (0.1%) | (100%) | |
| 11-16 | 5 mg | 104.9 mg | 15 mg | 0.125 mg | 125 mg | Size #2 |
| | (3%) | (83.9%)^{c} | (12.0%) | (0.1%) | (100%) | |
| 11-17 | 5 mg | 104.4 mg | 15 mg | 0.625 mg | 125 mg | Size #2 |
| | (3%) | (83.5%) | (12.0%) | (0.5%) | (100%) | |

^{a}PEG 3350 with high residual alkali (390 ppm sodium)
^{b}Powdered form of PEG 3350 (all others granular)
^{c}Powdered form of PEG 4000 (all others granular)

TABLE 12

| | Composition | | | | | |
|---|---|---|---|---|---|---|
| | | Solubilizing | Solubilizing | Citric | | Capsule |
| Formulation | Compound Ia | Agent | Agent | Acid | Total | Size |
| | | PEG 1450 | Gelucire | | | |
| | | | 44/14 | | | |
| 12-1 | 20 mg | 323.1 | 323.1 | 0.667 mg | 667 mg | Size #0 |
| | (3%) | (48.45%) | (48.45%) | (0.1%) | (100%) | |
| | | PEG 1450 | Tween 80 | | | |
| 12-2 | 20 mg | 486.2 mg | 160 mg | 0.667 mg | 667 mg | Size #0 |
| | (3%) | (72.9%) | (24.0%) | (0.1%) | (100%) | |
| 12-3 | 20 mg | 566.3 mg | 80 mg | 0.667 mg | 667 mg | Size #0 |
| | (3%) | (84.9%) | (12.0%) | (0.1%) | (100%) | |
| | | PEG 3350 | Gelucire | | | |
| | | | 44/14 | | | |
| 12-4 | 20 mg | 323.1 | 323.1 | 0.667 mg | 667 mg | Size #0 |
| | (3%) | (48.45%) | (48.45%) | (0.1%) | (100%) | |
| | | PEG 3350 | Tween 80 | | | |
| 12-5 | 20 mg | 486.2 mg | 160 mg | 0.667 mg | 667 mg | Size #0 |
| | (3%) | (72.9%) | (24.0%) | (0.1%) | (100%) | |
| 12-6 | 20 mg | 566.3 mg | 80 mg | 0.667 mg | 667 mg | Size #0 |
| | (3%) | (84.9%) | (12.0%) | (0.1%) | (100%) | |
| | | PEG 4000 | Gelucire | | | |
| | | | 44/14 | | | |
| 12-7 | 20 mg | 323.1 | 323.1 | 0.667 mg | 667 mg | Size #0 |
| | (3%) | (48.45%) | (48.45%) | (0.1%) | (100%) | |
| | | PEG 4000 | Tween 80 | | | |
| 12-8 | 20 mg | 486.2 mg | 160 mg | 0.667 mg | 667 mg | Size #0 |
| | (3%) | (72.9%) | (24.0%) | (0.1%) | (100%) | |
| 12-9 | 20 mg | 566.3 mg | 80 mg | 0.667 mg | 667 mg | Size #0 |
| | (3%) | (84.9%) | (12.0%) | (0.1%) | (100%) | |

TABLE 13

| | Composition | | | | | |
|---|---|---|---|---|---|---|
| | Compound | Solubilizing | solubilizing | Citric | | Capsule |
| Formulation | Ia | Agent | Agent | Acid | Total | Size |
| | | PEG 1450 | Gelucire | | | |
| | | | 44/14 | | | |
| 13-1 | 5 mg | 80.9 | 80.9 | 0.125 mg | 167 mg | Size #2 |
| | (3%) | (48.45%) | (48.45%) | (0.1%) | (100%) | |
| | | PEG 1450 | Tween 80 | | | |
| 13-2 | 5 mg | 121.7 mg | 40 mg | 0.125 mg | 167 mg | Size #2 |
| | (3%) | (72.9%) | (24.0%) | (0.1%) | (100%) | |
| 13-3 | 5 mg | 141.8 mg | 20 mg | 0.125 mg | 167 mg | Size #2 |
| | (3%) | (84.9%) | (12.0%) | (0.1%) | (100%) | |
| | | PEG 3350 | Gelucire | | | |
| | | | 44/14 | | | |
| 13-4 | 5 mg | 80.9 | 80.9 | 0.125 mg | 167 mg | Size #2 |
| | (3%) | (48.45%) | (48.45%) | (0.1%) | (100%) | |
| | | PEG 3350 | Tween 80 | | | |
| 13-5 | 5 mg | 121.7 mg | 40 mg | 0.125 mg | 167 mg | Size #2 |
| | (3%) | (72.9%) | (24.0%) | (0.1%) | (100%) | |
| 13-6 | 5 mg | 141.8 mg | 20 mg | 0.125 mg | 167 mg | Size #2 |
| | (3%) | (84.9%) | (12.0%) | (0.1%) | (100%) | |
| | | PEG 4000 | Gelucire | | | |
| | | | 44/14 | | | |
| 13-7 | 5 mg | 80.9 | 80.9 | 0.125 mg | 167 mg | Size #2 |
| | (3%) | (48.45%) | (48.45%) | (0.1%) | (100%) | |
| | | PEG 4000 | Tween 80 | | | |
| 13-8 | 5 mg | 121.7 mg | 40 mg | 0.125 mg | 167 mg | Size #2 |
| | (3%) | (72.9%) | (24.0%) | (0.1%) | (100%) | |
| 13-9 | 5 mg | 141.8 mg | 20 mg | 0.125 mg | 167 mg | Size #2 |
| | (3%) | (84.9%) | (12.0%) | (0.1%) | (100%) | |

### COMPARATIVE EXAMPLE 1

### Powder-in-Capsule

A mixture of Compound Ia anhydrous lactose at 90% by weight was filled into size #1 gray, opaque hard gelatin capsules and the capsules were encapsulated. Capsules were dosed to each of 2 dogs at a dose of approximately 2 mg/kg and plasma samples were taken and analyzed for pharmacokinetic parameters including drug concentrations versus time. Absolute oral bioavailability and coefficient of variation were determined as described above in Example 1.

| | | |
|---|---|---|
| Ingredient | Amount (mg) | Percentage of Total |

Comparative Composition 1

| | | |
|---|---|---|
| Compound Ia | 20.0 | 10.0% |
| Lactose, anhydrous | 180.0 | 90.0% |
| Total | 200.0 | 100.0% |

Pharmacokinetics

| | |
|---|---|
| F (Oral Bioavailability) | 2.7% |
| C.V. (Coefficient of | 7.4% |
| Variation) | |

### COMPARATIVE EXAMPLE 2

### Solution

Compound Ia was dissolved at 4 mg/mL in 10% Cremophor EL (cleaned by passage through an ion exchange resin)/10% Ethanol/80% Water and the solution was administered by oral gavage to each of 3 dogs at a dose of approximately 2 mg/kg. Plasma samples were taken and analyzed for pharmacokinetic parameters including drug concentrations versus time. The AUC's were calculated and used to determine the absolute oral bioavailability relative to Compound Ia administered intravenously to dogs from a PEG 400 solution.

| | |
|---|---|
| Ingredient | Amount |

Comparative Composition 2

| | | |
|---|---|---|
| Compound Ia | 4.0 | mg |
| Cremophor EL | 0.1 | mL |
| Ethanol | 0.1 | mL |
| Water | q.s. to 1.0 | mL |
| Total | 1.0 | mL |

Pharmacokinetics

| | |
|---|---|
| F (Oral Bioavailability) | 15.9% |
| C.V. (Coefficient of | 8% |
| Variation) | |

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departing from the scope of the appended claims.

What is claimed is:

1. A pharmaceutical composition comprising an orally-active taxane derivative having the formula: 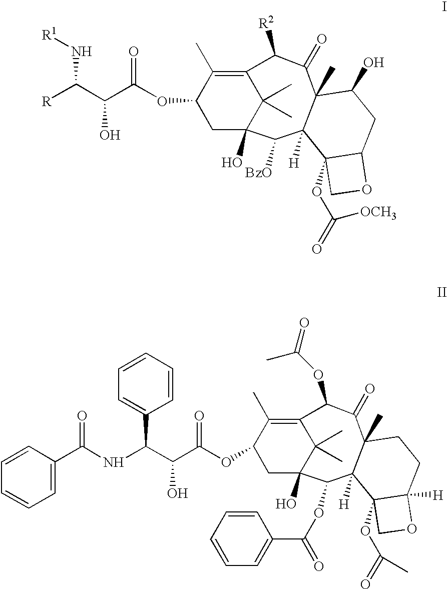
wherein:
R is phenyl, isopropyl, or tert butyl;
R¹ is —C(O)R^{z} in which R^{z} is (CH₃)₃CO—, (CH₃)₃CCH₂—, CH₃(CH₂)₃O—, cyclobutyl-, cyclohexyloxy, or (2-furyl); and
R² is CH₃C(O)O—, and a pharmaceutically acceptable solubulizing agent for said taxane derivative.

2. The composition as claimed in claim 1, wherein said compound is selected from the group consisting of compounds of formula I wherein R is selected from the group consisting of (CH₃)₃C—, (CH₃)₂CH—, and Phenyl-; R¹ is selected from the group consisting of (CH3)₃COC(O)—, (CH₃)₃CCH₂C(O)—, CyclobutylC(O)—, CyclohexylOC(O)—, CH₃(CH₂)₃OC(O)—, and (2-furyl)C(O)—; and R² is CH₃C(O)O—.

3. The composition as claimed in claim 1, comprising a compound of formula I in which R represents tert butyl; R¹ represents (CH₃)₃COC(O)—; and R² represents CH₃C(O)O—.

4. The composition as claimed in claim 1, comprising from about 1 to about 20 wt % of said taxane derivative and from about 10 to about 99 wt % of said solubulizing agent.

5. The composition as claimed in claim 1, wherein said solubulizing agent consists essentially of at least one solubilizer compound selected from the group consisting of a polyether glycol; a saturated or unsaturated polyglycolized glyceride; and a solid amphiphilic surfactant; and optionally, the composition further includes one or more of the group consisting of an alcohol other than a polyether glycol; a fatty acid ester derivatives of a polyhydric alcohol; a surfactant other than said solid amphiphilic surfactant; vegetable oil; and mineral oil.

6. The composition as claimed in claim 5, wherein said polyether glycol solubilizer compound is selected from the group consisting of a polyethylene glycol and a polyproplyene glycol and mixtures thereof.

7. The composition as claimed in claim 6, wherein said polyether glycol solubulizer compound comprises a polyethylene glycol.

8. The composition as claimed in claim 7, wherein the molecular weight of said polythylene glycol is in the range of 200-8000.

9. The composition as claimed in claim 5, wherein said polyglycolized glyceride solubilizer compound is saturated.

10. The composition as claimed in claim 5, wherein said solid amphiphilic surfactant solubulizer compound is selected from the group consisting of hydroxy-substituted stearic acid esters of polyethylene glycols and ∝-tocopheryl-polyethylene succinate esters of polyethylene glycols.

11. The composition as claimed in claim 5, wherein said fatty acid ester derivative of said polyhydric alcohol is selected from the group consisting of medium chain fatty acid monoglycerides, medium chain fatty acid diglycerides, medium chain fatty acid triglycerides and mixtures of said mono- di- and triglycerides.

12. The composition as claimed in claim 5, wherein said other surfactant is at least one surfactant selected from the group consisting of polyoxyethylene castor oil derivatives, polyoxethylene derivatives of fatty acid partial esters of sorbitan, polyoxyalkylene derivatives of propylene glycol, polyoxyethylene stearates, sorbitan fatty acid esters and lecithin.

13. The composition as claimed in claim 5, wherein said vegetable oil is selected from the group consisting of soybean oil, olive oil, peanut oil and sunflower oil.

14. The composition as claimed in claim 5, wherein said pharmacetically acceptable solubulizing agent consists essentially of polyethylene glycol as said solubilizer compound.

15. The composition as claimed in claim 14, wherein said solubilizer compound includes polyethylene glycol which is liquid at room temperature and polyethylene glycol which is solid at room temperature.

16. The composition as claimed in claim 14 further comprising at least one surfactant other than said solid, amphiphilic surfactant.

17. The composition as claimed in claim 5, wherein said pharmaceutically acceptable bioavailability enhancing agent consists essentially of saturated polyglycolized glyceride as said solubulizer.

18. The composition as claimed in claim 5, wherein said pharmaceutically acceptable solubulizing agent consists essentially of solid, amphiphilic surfactant as said solubulizer compound.

19. The composition as claimed in claim 5, wherein said solubilizer compound is solid at room temperature.

20. The composition as claimed in claim 5, wherein said solubilizer compound is liquid at room temperature.

21. The composition as claimed in claim 5, comprising said taxane derivative and a solubulizing agent comprising a plurality of said solubilizer compounds.

22. The composition as claimed in claim 21, wherein at least one of said plurality of solubilizer compounds is solid at room temperature and at least one other of said plurality of solubilizers is liquid at room temperature.

23. A composition as claimed in claim 21, wherein said solubilizer compound comprises at least one polyether glycol and at least one polyglycolized glyceride.

24. The composition as claimed in claim 21, wherein said solubilizer compound comprises at least one polyether glycol and at least one solid amphiphilic surfactant.

25. The composition as claimed in claim 21, wherein said composition comprises 4-10 wt % of said taxane derivative, 15-60 wt % of said polyether glycol; 15-60 wt % of said polyglycolized glyceride, 15-60 wt % of said solid amphiphilic surfactant and 5-40 wt % of a said other surfactant.

26. The composition as claimed in claim 1 or 14 in unit dosage form comprising, per unit, from about 2 mg to about 25 mg of said taxane derivative.

27. The composition as claimed in claim 26, wherein said unit dosage form is enclosed in a capsule.

28. The composition as claimed in claim 1, further comprising a pharmaceutically acceptable acid.

29. The composition as claimed in claim 28, wherein said pharmaceutically acceptable acid comprises citric acid.

30. The method of inhibiting tumor growth in a mammalian host which comprises administering to said mammal in need thereof a tumor-growth inhibiting amount of a composition as claimed in claim 1.

31. The method as claimed in claim 30, wherein the administration is oral.

32. A method for treatment of a cancer selected from the group consisting of ovarian, breast, brain, prostate, colon, stomach, kidney, and/or testicular cancer, Karposi's sarcoma; cholangiocarcinoma; choriocarcinoma; neuroblastoma; Wilm's tumor, Hodgkin's disease; melanomas; multiple myelomas; chronic lymphocytic leukemias; and acute or chronic granulocytic lymphomas in a patient in need of said treatment, said method comprising administering to said patient a pharmaceutical composition as claimed in claim 1.
